(19) United States
(12) Patent Application Publication
(10) Pub. No.: US 2022/0337762 A1
(43) Pub. Date: Oct. 20, 2022

(54) RADIATION IMAGING APPARATUS, RADIATION IMAGING SYSTEM, METHOD FOR CONTROLLING RADIATION IMAGING APPARATUS, AND STORAGE MEDIUM

(71) Applicant: CANON KABUSHIKI KAISHA, Tokyo (JP)

(72) Inventors: Toshitaka Noro, Saitama (JP); Asato Kosuge, Kanagawa (JP); Hideaki Morita, Tokyo (JP)

(21) Appl. No.: 17/722,237

(22) Filed: Apr. 15, 2022

(30) Foreign Application Priority Data
Apr. 20, 2021 (JP) 2021-071074

(51) Int. Cl. H04N 5/30; H04B 7/06

(52) U.S. Cl. CPC H04N 5/30; H04B 7/0608

(57) ABSTRACT

A radiation imaging apparatus includes a plurality of antennas that performs at least one of reception of control data for an image capturing unit to capture radiation image data from a data processing apparatus and transmission of the radiation image data to the data processing apparatus via wireless communication, a selection unit that selects an antenna to be used from the plurality of antennas, and a control unit that controls the selection unit. The control unit restricts a selection by the selection unit such that the selection is not performed during a period when the image capturing unit captures the radiation image data.

### BACKGROUND

### Field

The present disclosure relates to a radiation imaging apparatus, a radiation imaging system, a method for controlling a radiation imaging apparatus, and a storage medium.

### Description of the Related Art

Radiation imaging systems are increasingly digitalized due to the prevalence of a radiation imaging apparatus that captures a digital radiation image based on an emitted radiation. The digitalization of radiation imaging systems enables the confirmation of a radiation image immediately after the radiation image is captured. Thus, a workflow is significantly improved compared to image capturing using a conventional film or image capturing using a computed radiography (CR) apparatus. In recent years, a radiation imaging apparatus capable of performing wireless communication has improved the convenience of use of a radiation imaging apparatus.

As an example of a radiation imaging apparatus capable of performing wireless communication, Japanese Patent Application Laid-Open No. 2010-243486 discusses a radiation imaging apparatus on which a plurality of wireless communication antennas is mounted. The radiation imaging apparatus of Japanese Patent Application Laid-Open No. 2010-243486 is seen to select an antenna to be used from a plurality of wireless communication antennas based on a result of measuring the communication strength of each of the plurality of wireless communication antennas.

Japanese Patent Application Laid-Open No. 2010-243486 is not seen to discuss the timing when the antenna to be used is selected from the plurality of wireless communication antennas. There is room for considering the timing when an antenna is selected.

### SUMMARY

Aspects of the present disclosure are directed to providing a radiation imaging apparatus that selects an antenna to be used from a plurality of wireless communication antennas and performs selection control at a suitable timing.

According to an aspect of the present disclosure, a radiation imaging apparatus includes an image capturing unit configured to capture radiation image data based on emitted radiation, a plurality of antennas configured to perform at least one of reception of control data for the image capturing unit to capture the radiation image data from a data processing apparatus and transmission of the radiation image data to the data processing apparatus via wireless communication, a selection unit configured to select an antenna to be used from the plurality of antennas, and a control unit configured to control the selection unit. The control unit restricts selection such that the selection is not performed during a period when the image capturing unit captures the radiation image data.

Further features will become apparent from the following description of exemplary embodiments with reference to the attached drawings.

### BRIEF DESCRIPTION OF THE DRAWINGS

FIG. 1 is a conceptual block diagram illustrating an example of a radiation imaging system.

FIG. 2 is a conceptual block diagram illustrating an example of a radiation imaging apparatus.

FIG. 3 is a conceptual block diagram illustrating an example of an image capturing unit of the radiation imaging apparatus.

FIG. 4 is a conceptual block diagram illustrating an example of a wireless communication unit of the radiation imaging apparatus.

FIG. 5 is a flowchart illustrating control according to a first exemplary embodiment.

FIG. 6 is a timing chart illustrating the control according to the first exemplary embodiment.

FIG. 7 is a timing chart illustrating the control according to the first exemplary embodiment.

FIG. 8 is a flowchart illustrating control according to a second exemplary embodiment.

FIG. 9 is a timing chart illustrating the control according to the second exemplary embodiment.

### DESCRIPTION OF THE EMBODIMENTS

Exemplary embodiments will be discussed below with reference to the attached drawings. The following exemplary embodiments are not seen to be limiting. While a plurality of features is described in the exemplary embodiments, not all the plurality of features is essential, and the plurality of features can be optionally combined. In the attached drawings, the same or similar components are designated by the same reference numbers, and are not redundantly described. Examples of radiations according to the present disclosure include an α-ray, a β-ray, and a γ-ray, which are beams of particles (including photons) emitted by radiation decay, and also include beams having comparable or greater energy, such as an X-ray, a particle ray, and a cosmic ray.

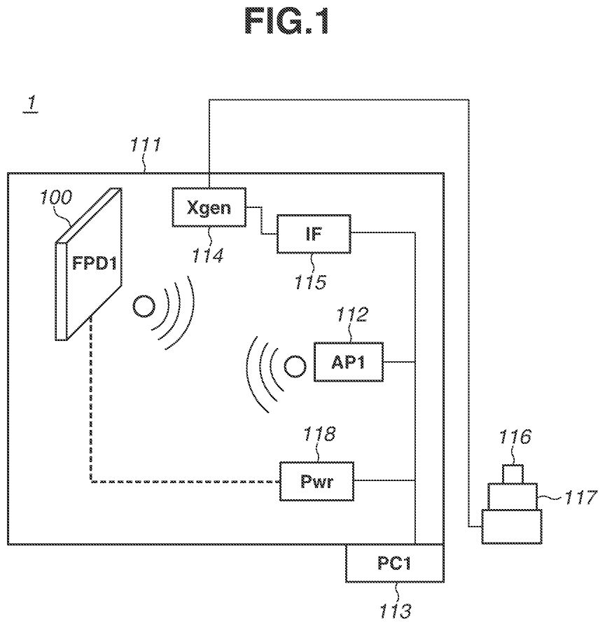

An example of the configuration of a radiation imaging system according to a first exemplary embodiment will now be described with reference to FIG. 1. A radiation imaging system 1 can be installed in a radiation imaging room 111. A radiation imaging apparatus 100 is moved and used in the radiation imaging room 111. The radiation imaging system 1 includes the radiation imaging apparatus 100, a wireless communication access point 112 (AP1), a console 113, a radiation generating apparatus 114, a relay 115, a radiation preparation request switch 116, a radiation emission request switch 117, and a power supply device 118. The console 113 is equivalent to a data processing apparatus and can control the AP1 112 and the radiation imaging apparatus 100. The relay 115 can adjust the timings of the radiation imaging apparatus 100 and the radiation generating apparatus 114. The radiation preparation request switch 116 can provide an instruction regarding a radiation emission preparation start request to the radiation generating apparatus 114. The radiation emission request switch 117 can provide a radiation emission instruction to the radiation generating apparatus 114. The radiation preparation request switch 116 and the radiation emission request switch 117 form a two-step switch, and the radiation preparation request switch 116 is always pressed before the radiation emission request switch 117. The power supply device 118 supplies power to the radiation imaging apparatus 100. The power supply device 118 includes an interface capable of supplying power and performing wired communication, and can relay communication between the radiation imaging apparatus 100 and communication devices. The radiation imaging apparatus 100 can be battery operated or operate via external power provided from the power supply device 118. The radiation imaging apparatus 100 can also transmit radiation image data to the console 113 through wireless communication via the AP1 112.

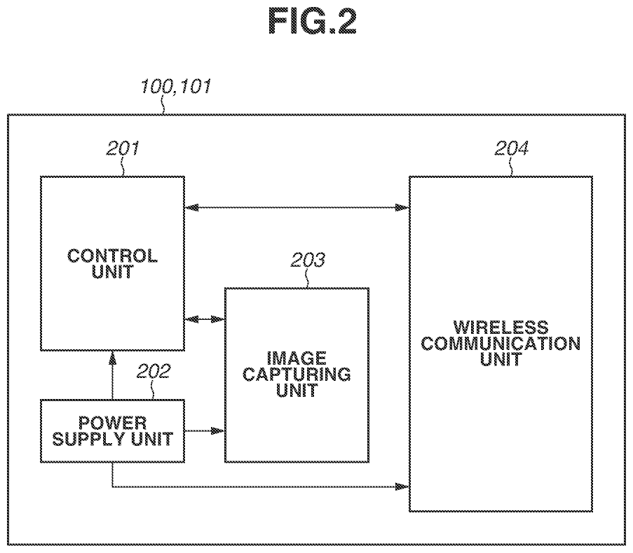

An example of the configuration of the radiation imaging apparatus 100 will now be described with reference to FIG. 2. The radiation imaging apparatus 100 includes a control unit 201, a power supply unit 202, an image capturing unit 203, and a wireless communication unit 204. The image capturing unit 203 can capture radiation image data based on an emitted radiation. The image capturing unit 203 includes a logic circuit such, as a field-programmable gate array (FPGA), a pixel array, and peripheral circuits of the pixel array. The detailed configuration of the image capturing unit 203 will be described below. The wireless communication unit 204 can receive control data for the image capturing unit 203 to capture an image from the console 113 and/or transmit the radiation image data to the console 113 via wireless communication. The wireless communication unit 204, which will be described in detail below, includes a plurality of antennas and is configured to perform selection control for selecting an antenna to be used from the plurality of antennas. The control unit 201 includes a central processing unit (CPU), memories such as a flash memory and a dynamic random-access memory (DRAM) as peripheral circuits of the CPU, and a wired communication interface. The control unit 201 controls the image capturing unit 203 and the wireless communication unit 204 or communicates with external devices. The power supply unit 202 supplies power to the control unit 201, the image capturing unit 203, and the wireless communication unit 204.

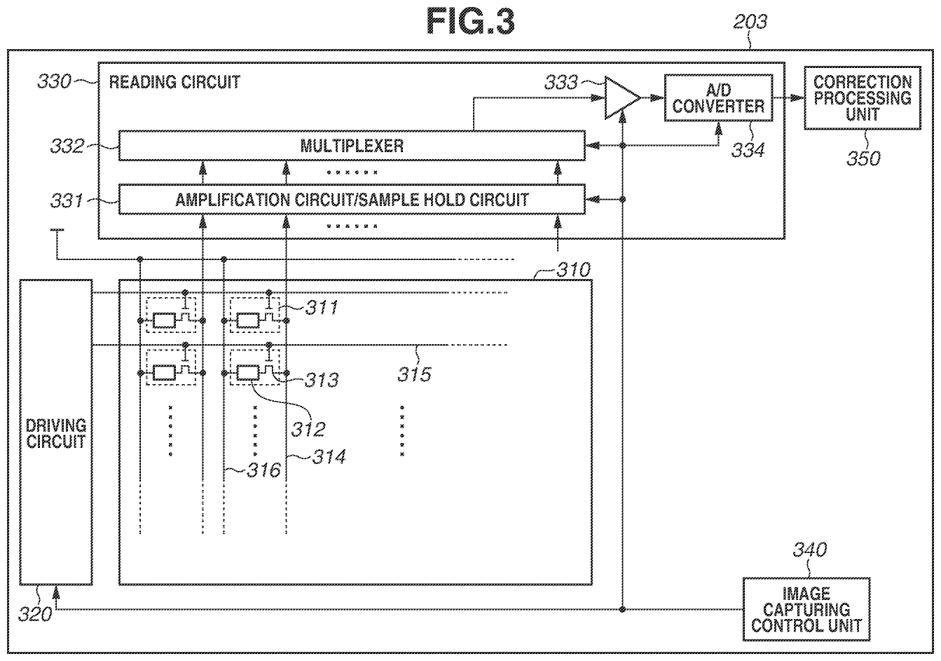

The configuration and the operation of the image capturing unit 203 will now be described with reference to FIG. 3. The image capturing unit 203 includes a pixel array 310, a driving circuit 320, and a reading circuit 330. The pixel array 310 includes a plurality of pixels 311 arranged in a two-dimensional array to form a plurality of rows and a plurality of columns. Each of the plurality of pixels 311 includes a conversion element 312 and a switch element 313. The conversion element 312 converts an incident radiation into an electric signal. The conversion element 312 can be composed of a scintillator that converts the radiation into visible light and a photoelectric conversion element that converts the visible light into an electric signal. In another exemplary embodiment, the conversion element 312 can be an element that directly converts the radiation into an electric signal. The switch element 313 transfers an electric signal accumulated in the conversion element 312 to a signal line 314. A transistor, such as a thin-film transistor (TFT), can be used as the switch element 313. The switch element 313 includes a control terminal. In response to the supply of an “on” voltage to the control terminal, the switch element 313 is turned on, i.e., enters a conducting state. In response to the supply of an “off” voltage to the control terminal, the switch element 313 is turned off, i.e., enters a non-conducting state. A bias voltage is supplied from the power supply unit 202 to one of terminals of the conversion element 312 via a bias line 316. The other terminal of the conversion element 312 is electrically connected to the signal line 314 via the switch element 313. The control terminal of the switch element 313 is connected to a driving line 315. In the pixel array 310, a plurality of driving lines 315 extending in the row direction (the horizontal direction in FIG. 3) is located next to each other in the column direction (the vertical direction in FIG. 3). The control terminals of the switch elements 313 of pixels 311 included in the same row are commonly connected to each driving line 315. In the pixel array 310, a plurality of signal lines 314 extending in the column direction is also located next to each other in the row direction. One of the main terminals of each of the switch elements 313 of pixels 311 included in the same column is commonly connected to each signal line 314.

The driving circuit 320 drives the pixel array 310 based on a control signal supplied from the control unit 201. Specifically, the driving circuit 320 supplies a driving signal to the control terminal of each switch element 313 via the driving line 315. The driving circuit 320 changes the driving signal to an “on” voltage, thereby turning on the switch element 313. The driving circuit 320 changes the driving signal to an “off” voltage, thereby turning off the switch element 313. If the switch element 313 is turned on, an electric signal accumulated in the conversion element 312 is transferred to the signal line 314.

Based on a control signal supplied from a control unit 340 that can be included in the control unit 201, the reading circuit 330 reads electric signals from the pixel array 310 and outputs radiation image data based on the electric signals. The output radiation image data is supplied to a correction processing unit 350. The reading circuit 330 includes an amplification circuit/sample hold circuit 331, a multiplexer 332, an amplifier 333, and an analog-to-digital (A/D) converter 334. The amplification circuit/sample hold circuit 331 amplifies electric signals read from the conversion elements 312 and samples and holds the electric signals in units of pixel rows. The multiplexer 332 sequentially extracts electric signals of pixels in a single row that are held in the amplification circuit/sample hold circuit 331 and supplies the electric signals to the amplifier 333. The amplifier 333 amplifies the supplied electric signals and supplies the amplified electric signals to the A/D converter 334. The A/D converter 334 converts the supplied analog electric signals into digital signals (equivalent to the above-described radiation image data) and supplies the digital signals to the correction processing unit 350.

The correction processing unit 350 can perform offset correction for subtracting offset image data acquired from the pixel array 310 without emitting radiation from the radiation image data and obtain radiation image data from which an unnecessary offset component is removed.

Both the driving circuit 320 and the reading circuit 330 can be composed of a programmable logic circuit such as an FPGA or a complex programmable logic device (CPLD), a dedicated integrated circuit (IC), and a printed circuit board on which the programmable logic circuit and the dedicated IC are mounted.

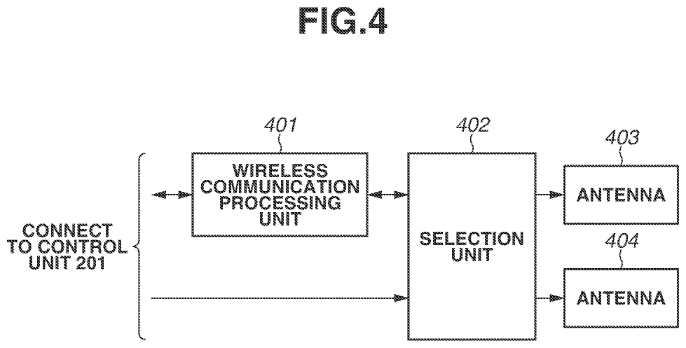

The configuration of the wireless communication unit 204 will now be described with reference to FIG. 4. The wireless communication unit 204 includes a wireless communication processing unit 401, a selection unit 402, a first antenna 403, and a second antenna 404. While FIG. 4 illustrates a configuration in which two antennas are used for description purposes, in additional exemplary embodiments, a configuration can be employed where more than two antennas are mounted.

The wireless communication processing unit 401 can be a one-way communication device that receives control data from the control unit 201 via the first antenna 403 or the second antenna 404, or can be a two-way communication device that transmits and receives control data and radiation image data.

The wireless processing unit 401 can use any appropriate wireless communication protocol, such as the Bluetooth® communication standard. The wireless processing unit 401 can also use any Institute of Electrical and Electronics Engineers (IEEE) 802.11 communication standard or any other wireless communication standard.

The selection unit 402 selects an antenna to be used from the plurality of antennas. Specifically, the selection unit 402 has an analog switch configuration for, based on an instruction from the control unit 201, performing selection control for selecting the first antenna 403 or the second antenna 404. More specifically, the selection unit 402 implements the following process. If the voltage level of a signal from the control unit 201 is a ‘low’ level (a ground (GND) level), the first antenna 403 is selected. If the voltage level is a ‘high’ level (a power supply voltage level), the second antenna 404 is selected. The antenna selection control is not limited to a configuration in which a single antenna is selected from among the plurality of antennas. Additional exemplary embodiments can include, configurations with two or more antennas selected from among the plurality of antennas.

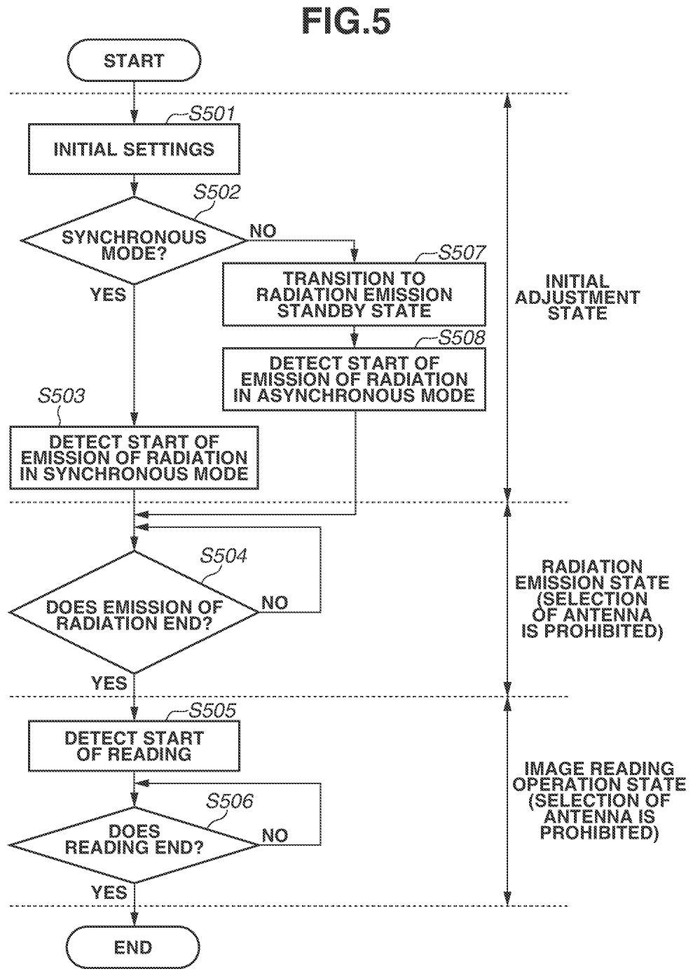

The antenna selection control will now be described with reference to FIG. 5. The image capturing method of the radiation imaging system 1 can have two image capturing modes, each described below. The two image capturing modes can be set through the console 113 by an operator. Information regarding these image capturing modes is set as image capturing information in the radiation imaging apparatus 100 via the console 113 before a radiation is emitted.

The first image capturing mode is a synchronous mode where the relay 115 and the radiation imaging apparatus 100 communicate a synchronous signal to each other, thereby synchronizing the operations of the radiation imaging apparatus 100 and the radiation generating apparatus 114. Based on a synchronous signal received by the control unit 201 of the radiation imaging apparatus 100 via the relay 115 or the wireless communication access point 112, the control unit 201 detects the start of emission of radiation from the radiation generating apparatus 114. This synchronous signal is a signal indicating that the radiation generating apparatus 114 generates radiation. The control unit 201 then controls, with reference to the timing of the detection, the image capturing unit 203 based on a radiation emission time included in the image capturing information set in advance by an operator.

The second image capturing mode is an asynchronous mode where the relay 115 and the radiation imaging apparatus 100 do not require a synchronous signal, and the operations of the radiation imaging apparatus 100 and the radiation generating apparatus 114 are not synchronized with each other. A radiation emitted from the radiation generating apparatus 114 is converted into an electric signal by the image capturing unit 203. The control unit 201 detects, as the start of emission of radiation, for example, the timing when the value of the electric signal from the image capturing unit 203 exceeds a threshold set in advance. Similarly to the synchronous mode, the control unit 201 controls, with reference to the timing of the detection, the image capturing unit 203 based on the radiation emission time included in the image capturing information set in advance by the operator. That is, in the asynchronous mode, the control unit 201 functions as a detection unit that detects the start of emission of radiation.

The antenna selection control will now be described. The antenna selection control can be performed for the purpose of ensuring wireless communication quality. For example, when the wireless communication quality decreases due to a change in phase or intensity under the influence of the distance between transmission and reception or an obstacle, selection control for selecting another antenna is performed. The antenna selection control, however, involves current consumption, and a fluctuation in an electric field or a magnetic field due to such current consumption influences the radiation imaging apparatus 100. Moreover, simultaneously with the antenna selection control, an error between a transmission process and a reception process performed by each antenna, an error between transmission processes performed by the plurality of antennas, or an error between reception processes performed by the plurality of antennas can be corrected (calibrated). Such calibration can further consume a large current. The present inventor has found that a current fluctuation involved in such current consumption has a great influence when radiation is emitted or while a reading operation for reading an image from the reading circuit 330 is performed, and therefore the current fluctuation can be the cause of noise.

In response, the present inventor has found that it is effective if the antenna selection control is performed at an appropriate timing, and the selection of an antenna is restricted so that an antenna is not selected during the period when the image capturing unit 203 captures an image. The present inventor has found that it is more desirable that if the selection of an antenna is restricted by prohibiting the selection of an antenna during the period when the image capturing unit 203 captures an image. The timing of the prohibition of the antenna selection control according to the present exemplary embodiment will now be described with reference to a flowchart illustrated in FIG. 5. This processing can be executed based on an instruction from the control unit 201.

In step S501, the control unit 201 is in an initial adjustment state. In the initial adjustment state, the control unit 201 receives image capturing information (control data) via the power supply device 118 or the wireless communication access point 112 and stores the image capturing information (the control data) in a memory. The image capturing information (the control data) includes an image capturing mode, an emission time, and the time of a reading operation for reading radiation image data that are set through the console 113 by an operator. The control unit 201 executes the following image capturing operation based on the stored image capturing information.

In step S502, the control unit 201 determines the image capturing mode based on the stored image capturing information. If it is determined that the image capturing mode is the synchronous mode (YES in step S502), the processing proceeds to step S503. If it is determined that the image capturing mode is the asynchronous mode (NO in step S502), the processing proceeds to step S507.

In step S503, the control unit 201 detects a synchronous signal. The synchronous signal is transmitted from the relay 115 to the control unit 201 via the power supply device 118 or the wireless communication access point 112 when the operator presses the radiation emission request switch 117. The control unit 201, upon receipt of the synchronous signal, transitions to a radiation emission state. The processing then proceeds to step S504.

In step S507, the control unit 201 causes the radiation imaging apparatus 100 to transition to a standby state where the emission of radiation from the radiation generating apparatus 114 is permitted. The “standby state” is a state where the radiation imaging apparatus 100 can detect the start of emission of radiation.

In step S508, if the operator presses the radiation emission request switch 117, the radiation generating apparatus 114 emits radiation. The emitted radiation is converted into an electric signal by the image capturing unit 203. The control unit 201 detects, for example, the start of emission of radiation as the timing when the value of the electric signal from the image capturing unit 203 exceeds a pre-set threshold. The control unit 201 uses the detected start of emission of radiation as a trigger to transition to a radiation emission state. The processing then proceeds to step S504.

In step S504, the control unit 201 maintains the radiation imaging apparatus 100 in the emission state until the radiation emission time included in the stored image capturing information elapses. The emission state is the state where the radiation imaging apparatus 100 can receive the emission of radiation to capture radiation image data. While the radiation imaging apparatus 100 is in the emission state, the radiation generating apparatus 114 emits radiation to the radiation imaging apparatus 100. The control unit 201 enters a selection prohibition state during the period of the emission state from the start of the emission state until the emission time elapses. The selection prohibition state is a state where the control unit 201 does not provide an instruction to perform the antenna selection control to the selection unit 402. The control unit 201 thereby restricts the selection of an antenna so that an antenna is not selected during the period of the emission state.

In step S505, the control unit 201 instructs the reading circuit 330 to start a reading operation for reading electric signals from the pixel array 310. The reading operation is started based on an instruction from the control unit 201 at a timing based on pre-set image capturing information. If the control unit 201 determines the start of the reading operation, the control unit 201 causes the radiation imaging apparatus 100 to transition to a reading operation state.

In step S506, the control unit 201 maintains the radiation imaging apparatus 100 in the reading operation state from the timing of the start of the reading in step S505 to the lapse of the time of the reading operation included in the stored image capturing information.

In steps S505 and S506, the control unit 201, upon the start of the reading operation, is in a selection prohibition state during the period of the reading operation state from the start of the reading operation to the lapse of the time of the reading operation. The control unit 201 thereby restricts the selection of an antenna so that an antenna is not selected during the period of the reading operation state.

In step S506, the control unit 201 ends the selection prohibition state and removes the restriction of the selection of an antenna in response to the lapse of the time of the reading operation and the end of the reading operation state. That is, the control unit 201 ends the prohibition of the selection of an antenna in response to the end of the output of the radiation image data from the reading circuit 330. The control unit 201 thereby ends the prohibition of the selection of an antenna in response to the end of the capturing of the radiation image data by the image capturing unit 203.

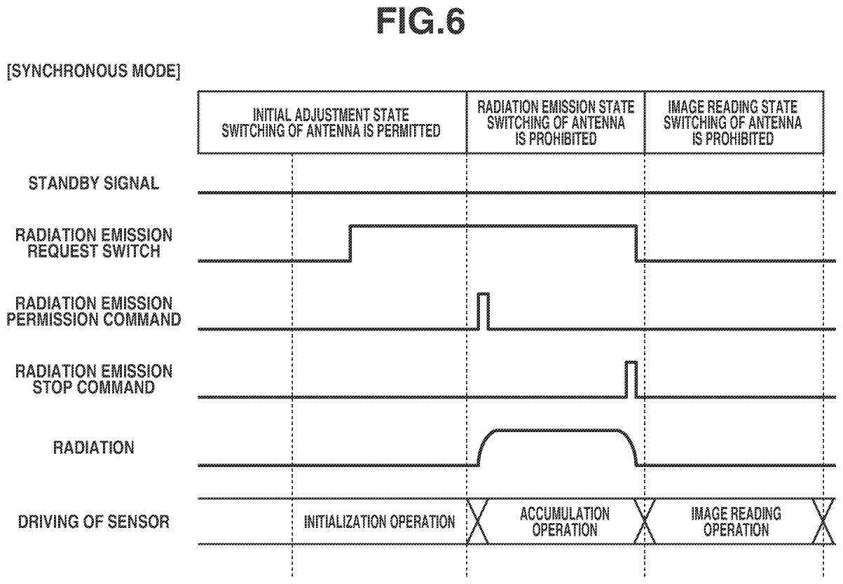

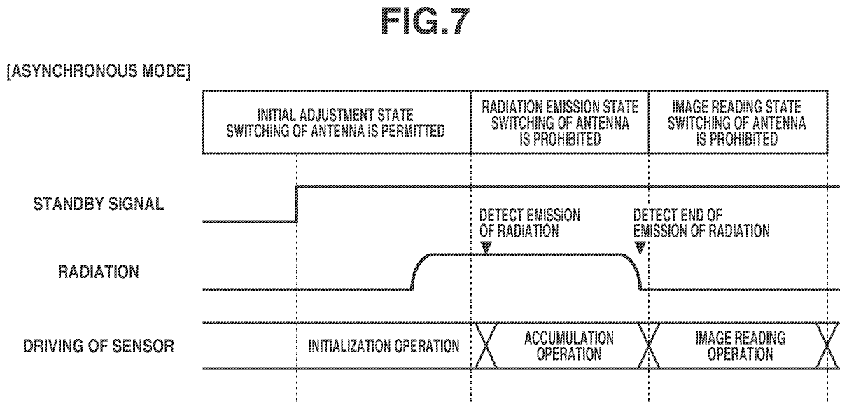

Control of the radiation imaging system 1 is described with reference to FIGS. 6 and 7. To execute the flow illustrated in FIG. 5, the radiation imaging system 1 can be controlled by using control signals illustrated in FIGS. 6 and 7.

In the synchronous mode illustrated in FIG. 6, the radiation emission request switch 117 is pressed, resulting in the control unit 201 generating a synchronous signal based on a signal of the radiation emission request switch 117 and enters a selection prohibition state. The radiation generating apparatus 114 receives the generated synchronous signal and notifies the control unit 201 of a radiation stop command. In the asynchronous mode illustrated in FIG. 7, the control unit 201 enters a selection prohibition state in response to the start of emission of radiation based on a detection signal indicating the detection of the start of emission. The control unit 201 also enters a selection prohibition state in response to the start of a reading operation, and the selection prohibition state is cancelled in response to the end of the reading operation.

As described above, an antenna selection prohibition state is provided in antenna selection control, which enables reducing a cause of noise due to a current fluctuation.

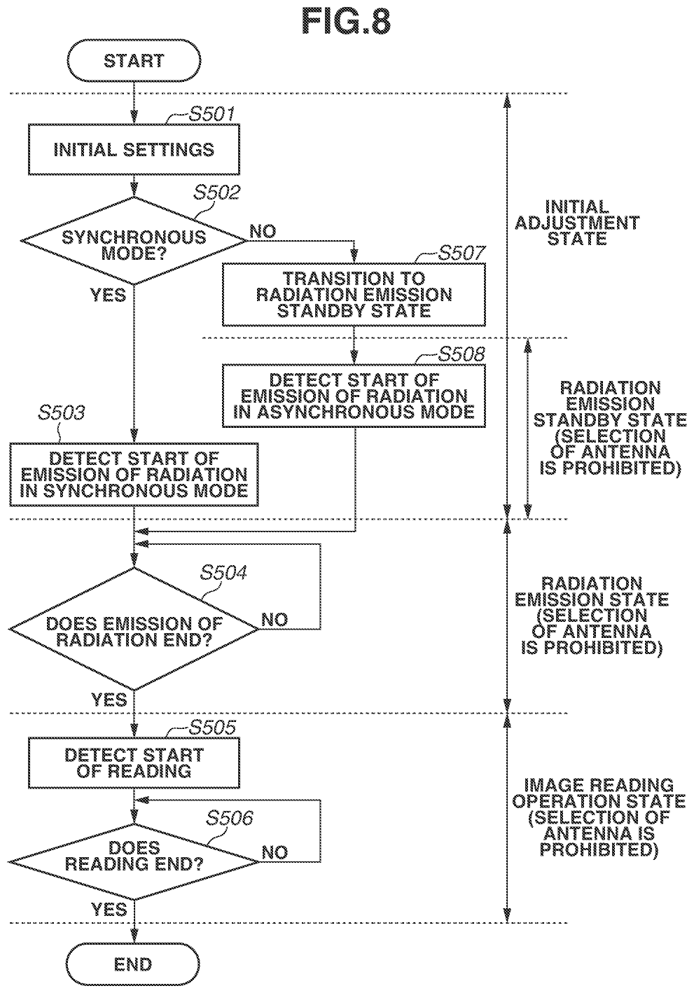

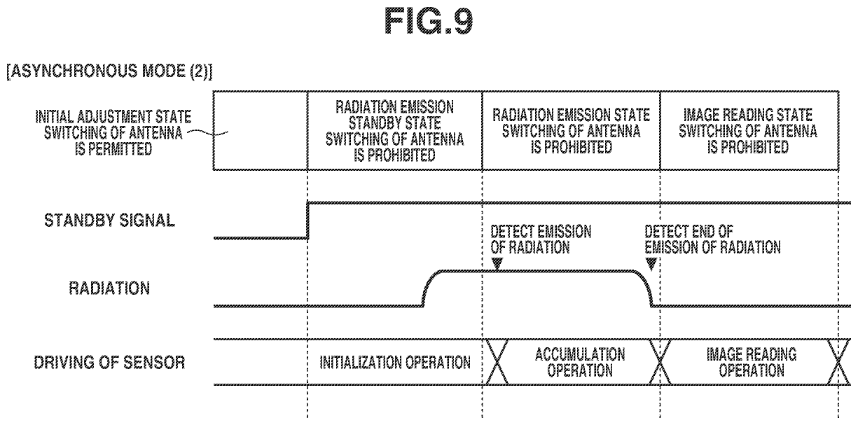

A second exemplary embodiment will now be described with reference to FIGS. 8 and 9. In the second exemplary embodiment, the differences from the first exemplary embodiment are described, while configurations and operations similar to those in the first exemplary embodiment are designated by the same names and signs, and as such, are not described in detail below.

In a case where the control unit 201 detects the start of emission of radiation in the asynchronous mode according to the first exemplary embodiment, a current fluctuation related to the antenna selection control can influence electric signals of the pixel array 310 for detecting the start of emission of radiation. This can cause the control unit 201 to erroneously detect the start of emission of radiation.

In the second exemplary embodiment, the control unit 201 enters, in the asynchronous mode, a selection prohibition state based on the radiation imaging apparatus 100 entering a standby state in step S507, as illustrated in FIG. 8. Specifically, the control unit 201 starts prohibiting the selection of an antenna upon receipt of a standby detection signal, as illustrated in FIG. 9. The standby detection signal indicates that the detection of the start of emission of radiation is to be started. It is thereby possible to reduce a decrease in the accuracy of detecting the start of emission of radiation in addition to the reduction in the cause of noise due to a current fluctuation and to provide a stable image and stable radiation detection accuracy.

The above-described exemplary embodiments can also be achieved by performing the process of supplying software (a program) for achieving the functions of the above exemplary embodiments to a system or an apparatus via a network or a storage medium, and causing a computer (or a CPU or a microprocessor unit (MPU)) of the system or the apparatus to read and execute the program.

### (Other Embodiments)

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD™), a flash memory device, a memory card, and the like.

The above-described exemplary embodiments are not seen to be limiting, and can be changed and modified without departing from the spirit and the scope of the present disclosure.

According to aspects of the present disclosure, a radiation imaging apparatus is provided that selects an antenna to be used from a plurality of wireless communication antennas and performs selection control at a suitable timing.

While exemplary embodiments have been provided, these embodiments are not seen to be limiting. The scope of the following claims is to be accorded the broadest interpretation so as to encompass all such modifications and equivalent structures and functions.

This application claims the benefit of Japanese Patent Application No. 2021-071074, filed Apr. 20, 2021, which is hereby incorporated by reference herein in its entirety.

What is claimed is:

1. A radiation imaging apparatus comprising:
an image capturing unit configured to capture radiation image data based on emitted radiation;
a plurality of antennas configured to perform at least one of reception of control data for the image capturing unit to capture the radiation image data from a data processing apparatus and transmission of the radiation image data to the data processing apparatus via wireless communication;
a selection unit configured to select an antenna to be used from the plurality of antennas; and
a control unit configured to control the selection unit,
wherein the control unit restricts selection by the selection unit such that the selection is not performed during a period when the image capturing unit captures the radiation image data.

2. The radiation imaging apparatus according to claim 1, wherein the control unit, in response to receipt of a signal indicating that a radiation generating apparatus configured to generate the radiation generates the radiation, begins to restrict the selection.

3. The radiation imaging apparatus according to claim 1, further comprising a detection unit configured to detect a start of emission of the radiation,
wherein, in response to a signal indicating that the detection unit detects the emission of the radiation, the control unit starts restricting the selection.

4. The radiation imaging apparatus according to claim 1, further comprising a detection unit configured to detect a start of emission of the radiation,
wherein, in response to receipt of a signal indicating that the detection unit is to start detecting the start of the emission of the radiation, the control unit starts restricting the selection.

5. The radiation imaging apparatus according to claim 1, wherein, in response to an end of a capturing of the radiation image data by the image capturing unit, the control unit ends restricting the selection.

6. The radiation imaging apparatus according to claim 1,
wherein the image capturing unit includes a pixel array in which a plurality of pixels, each including a conversion element configured to convert a radiation into an electric signal, is arranged in a two-dimensional array, a driving circuit configured to drive the pixel array, and a reading circuit configured to output the radiation image data based on electric signals read from the pixel array driven by the driving circuit, and
wherein the control unit controls the driving circuit and the reading circuit.

7. The radiation imaging apparatus according to claim 6, wherein, in response to an end of the output of the radiation image data from the reading circuit, the control unit ends the restriction of the selection.

8. A radiation imaging system comprising:
the radiation imaging apparatus according to claim 1; and
the data processing apparatus.

9. A method for controlling a radiation imaging apparatus including a plurality of antennas, the method comprising:
capturing radiation image data based on emitted radiation;
performing, via the plurality of antennas least one of reception of control data for the image capturing unit to capture the radiation image data from a data processing apparatus and transmission of the radiation image data to the data processing apparatus via wireless communication; and
selecting an antenna to be used from the plurality of antennas,
wherein selecting an antenna is restricted such that the selection is not performed during a period of capturing the radiation image data.

10. A storage medium that stores a program for causing a computer to execute the control method for controlling the radiation imaging apparatus according to claim 9.
